(19) United States
(12) Plant Patent Application Publication
(10) Pub. No.: US 2026/0083033 P1
(43) Pub. Date: Mar. 19, 2026

(54) Miniature Rose Plant Named 'poulpal097'

(50) ;

(71) Applicant: Mogens Nyegaard Olesen, Fredensborg (DK)

(72) Inventor: Mogens Nyegaard Olesen, Fredensborg (DK)

(73) Assignee: poulsen Roser A/S, Fredensborg (DK)

(21) Appl. No.: 18/831,801

(22) Filed: Sep. 15, 2025

(30) Foreign Application Priority Data
Sep. 19, 2024 (QZ) 20242187

(51) Int. Cl. A01H 6/74; A01H 5/02

(52) U.S. Cl. CPC A01H 6/749; A01H 5/02
USPC PLT/116; PLT/122

(57) ABSTRACT

A new garden rose plant of the miniature class which has abundant, red flowers and attractive foliage. This new and distinct variety has shown to be uniform and stable in the resulting generations from asexual propagation.

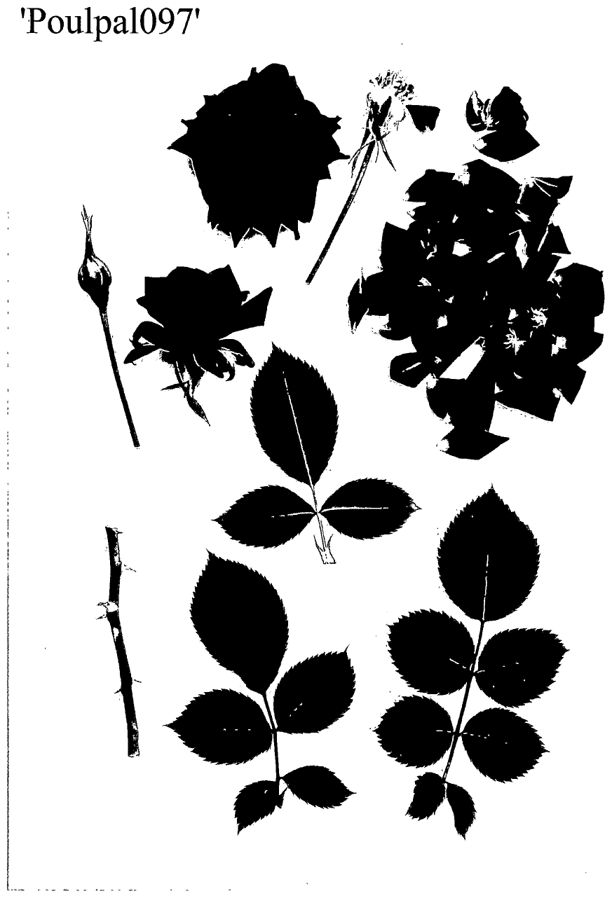

Botanical designation: Rosa hybrida.

Variety denomination: ‘Poulpal097’.

This application claims priority to Plant Breeder's Rights Application Number 2024/2187, which was filed at the Community Plant Variety Rights Office in the European Union on Sep. 19, 2024, the contents of which are hereby incorporated by reference for all purposes.

### SUMMARY OF THE INVENTION

The present invention constitutes a new and distinct variety of rose plant which originated from a controlled crossing between the female seed parent, an unnamed seedling, and the male pollen parent, also an unnamed seedling. Both of the parent varieties are non-patented.

The two parents were crossed during the summer of 2013 and the resulting seeds were planted in a controlled environment in Odense, Denmark. The new variety, named ‘Poulpal097’, originated as a single seedling from the stated cross.

The new variety may be distinguished from its male pollen parent and female seed parent primarily by the following characteristics. The male pollen parent plant has Red-Purple Group N 57A flowers while the new variety has Red Group 46B flowers. The female seed parent plant has a climbing rose growth habit while the new variety has a compact floribunda growth habit.

The objective of the hybridization of this rose variety was to create a new and distinct variety with unique qualities, such as:
1. Uniform and abundant red flowers;
2. Vigorous, but compact growth when propagated on its own roots;
3. Suitability for production in containers in open air nurseries; and
4. Exceptional disease resistance.

This combination of qualities is not present in previously available commercial cultivars of this type, known to the inventor, and distinguish ‘Poulpal097’ from all other varieties of which we are aware.

As part of the rose development program, Mogens N. Olesen germinated the seeds from the aforementioned hybridization during winter of 2013 and conducted evaluations on the resulting seedlings in a controlled environment in Odense, Denmark. ‘Poulpal097’ was selected in the spring of 2014 by the inventor as a single plant from the progeny of the aforementioned hybridization.

Asexual reproduction of ‘Poulpal097’ by rooted cuttings was first done by Mogens N. Olesen in the nursery in Odense, Denmark in July 2014. This initial and other subsequent asexual propagations conducted in controlled environments have demonstrated that the characteristics of ‘Poulpal097’ are true to type and are transmitted from one generation to the next.

### DESCRIPTION OF THE DRAWING

The accompanying color illustration shows as true as is reasonably possible to obtain in color photographs of this type, the typical characteristics of the buds, flowers, leaves, and stems, of ‘Poulpal097’. Specifically illustrated in the drawing are open flowers viewed from the side and above, flower petals detached revealing reproductive flower parts, a flower bud prior to opening, bare stem exhibiting prickles, juvenile leaves demonstrating the color of anthocyanin, and mature compound leaves. Plants shown are 3 months of age.

### DETAILED DESCRIPTION OF THE VARIETY

The following is a description of ‘Poulpal097’,as observed in its growth in a glass house located in Odense Denmark. Observed plants are 3 months of age, and were grown on their own roots in 17 cm containers. Color references are made using The Royal Horticultural Society (London, England) Colour Chart, 2001, except where common terms of color are used.

For a close comparison, several physical characteristics of the rose variety ‘Poulpal059’, U.S. Plant Pat. No. 27,739 are compared to the claimed plant. While ‘Poulpal097’ has about 100 flower petals, ‘Poulpal059’ has 35 to 40 flower petals. The claimed plant has a flower diameter of 60 mm while ‘Poulpal059’ has a flower diameter of 55 mm. Open flowers of ‘Poulpal097’ are generally Red Group 46B in color while ‘Poulpal059’ has a general tonality of Red Group 46A.

### Flower and Flower Bud

Blooming habit: Continuous.
Flower bud:
   Size.—Upon opening, 21 mm in length from base of receptacle to end of bud. Bud diameter is 12 mm.
   Bud form.—Globose.
   Bud color.—As sepals divide petals are Red Group 46A.
   Sepal inner surface.—Color: Yellow-Green Group 144A. Surface: Lightly pubescent.
   Sepal outer surface.—Color: Yellow-Green Group 144A. Texture: Smooth.
   Sepal shape.—Apex: Cirrhose. Base: Flat at union with receptacle.
   Sepal margin.—Margins have weak foliaceous appendages on three of the five sepals.
   Sepal size.—30 mm long, 6 mm wide.
   Receptacle.—Texture: Smooth. Size: 7 mm in height, 7 mm wide. Color: Yellow-Green Group 144A. Shape: Funnel.
   Pedicel.—Surface: Smooth. Length: 65 mm. Diameter: 2 mm on average. Color: Yellow-Green Group 144A. Strength: Strong.
   Peduncle.—Length: 20 to 50 mm. Diameter: About 4 mm. Color: Yellow-Green Group 144A. Texture: Smooth.
Flower bud development: Flower buds are borne in clusters of Up to 3, or single.
Flower bloom:
   Fragrance.—Moderate perfume.
   Duration.—The blooms have a duration on the plant of approximately 21 days. Petals fall cleanly away from plant after flowers have fully matured.
   Size.—Flower diameter is 60 mm when open. Flower depth is 24 mm.
   Flower shape.—Star shaped, with a high pointed center which is tightly closed.
   Shape of flower, side view.—The upper portion convex. The lower portion is flat concave.
Petalage: Under normal conditions, flowers have about 100 petals.
General tonality of flower: Open flowers are Red Group 46B.
Petal color: Upon opening, and after opening outer & inner petals. Upper surface: Red Group 53A. Lower surface: Red Group 53C, with intonations of Red Group 53B at the marginal zone. Basal petal spots, upon and after opening: Upper surface: Yellow Group 4A. Lower surface: Yellow Group 4C. After opening, outer petals Upper surface: Red Group 53B. Lower surface: Red Group 54A with intonations of Red Group 53B at the marginal zone.
Petals:
   Petal reflex.—Very strong, bilateral.
   Margin.—Entire and uniform. No undulation.
   Shape.—Broad and elliptic. Apex shape: Rounded. Base shape: Acute.
   Size.—31 mm (l)×30 mm (w).
   Texture.—Smooth.
   Thickness.—Average.
Petaloids:
   Size.—15 mm (l) by 6 mm (w).
   Quantity.—About 24.
   Shape.—Elliptical with an acute base and rounded apices.
   Color.—Upper surface is Red Group 53A with intonations of Red-Purple Group 59B. At the petaloid base Yellow Group 4B. Lower Red-Purple Group 59C.
Reproductive flower parts:
   Pollen.—None observed.
   Anthers.—Size: 2 mm in length. Color: Greyed-Orange Group 165A. Quantity: 40 on average.
   Filaments.—Color: Yellow Group 5B. Length: 4 mm.
   Pistils.—Length: 10 mm. Quantity: 15 on average.
   Stigmas.—Color: Yellow-White Group 158D.
   Styles.—Color: Green-White Group 157A with intonations of Red-Purple Group 60C.
   Location of stigmas.—Superior in location relative to the length of the filaments and the height of the anthers.
   Hips.—None Observed. Plant
Plant growth: Upright. Plants are about 40 cm in height, and 30 cm wide.
Stems:
   Color of juvenile growth.—Yellow-Green Group 144B.
   Color of mature growth.—Yellow-Green Group 144A.
   Length.—Canes are about 20 cm from the base of the plant to the flowering portion.
   Diameter.—About 4 mm.
   Internodes.—On mature canes about 27 mm between nodes.
   Surface texture.—Young wood: Smooth. Older wood: Smooth.
Long prickles:
   Incidence: 10 prickles per 10 cm of stem.
   Size.—Average length of prickles on mature stems is 6 mm.
   Shape.—Upper portion is linear. Lower portion is concave.
   Color.—Juvenile prickles: Greyed-Yellow Group 160 C Mature prickles: Greyed-Yellow Group 160 C
Plant foliage:
   Compound leaf.—122 mm (l)×74 mm (w).
   Quantity.—3 leaves per 10 cm of stem on average.
   Leaf bearing angle to the stem.—45 to 70 degrees.
   Color of juvenile foliage.—Upper side: Yellow-Green Group 146B with intonations of Greyed-Orange Group 166A. Lower side: Yellow-Green Group 146C with intonations of Greyed-Orange Group 166A.
   Color of mature foliage.—Upper side: Yellow-Green Group 146A. Lower side: Yellow-Green Group 146B.
Plant leaves and leaflets:
   Stipules.—Size: 4 mm long, 2 mm wide. Quantity: 2 per compound leaf. Shape: Linear, slightly broad based with outward extending apices. Margins: Finely serrated. Color: Yellow-Green Group 144A.
   Petiole.—Length: 10 mm. Diameter: 2 mm. Upper surface color: Yellow-Green Group 144A with intonations of Greyed-Purple Group 183C. Lower surface color: Yellow-Green Group 144A.
   Rachis.—Length: About 60 mm. Upper surface color: Yellow-Green Group 144A with intonations of Greyed-Purple Group 183C. Lower surface color: Yellow-Green Group 144A.
   Leaflet.—Quantity: Normally 5 to 7 leaflets. Margins: Serrated. Size: Terminal leaflets are about 54 mm long, 36 mm wide. Shape: Generally elliptical. Base: Rounded. Apex: Mucronate. Texture: Smooth. Thickness: Average. Arrangement: Odd pinnate. Venation: Reticulate. Glossiness: Glossy.
Disease resistance: Above average resistance to powdery mildew Sphaerotheca pannosa var. rosae, downy mildew Peronospora sparsa, rust Phragmidium spp., black spot Diplocarpon rosae, and Botrytis cinerea under normal growing conditions.
Cold hardiness: The variety is tolerant to USDA Cold Hardiness Zone 6.
Heat tolerance: The variety has been found to be suitable for climate conditions found in the American Horticulture Society heat zone 7.

I claim:

1: A new and distinct variety of rose plant named ‘Poulpal097’ substantially as described and illustrated herein.
